(19) United States
(12) Patent Application Publication
(10) Pub. No.: US 2019/0029573 A1
(43) Pub. Date: Jan. 31, 2019

(54) BIOLOGICAL INFORMATION DISPLAY APPARATUS

(71) Applicant: DENSO CORPORATION, Kariya-city, Aichi-pref. (JP)

(72) Inventors: Rie OSAKI, Kariya-city (JP); Shinya KUROSAWA, Kariya-city (JP); Kazuo TOKUSHIMA, Kariya-city (JP)

(21) Appl. No.: 16/075,692

(22) PCT Filed: Feb. 8, 2017
(86) PCT No.: PCT/JP2017/004642

§ 371 Date: Aug. 6, 2018

(30) Foreign Application Priority Data
Feb. 8, 2016 (JP) 2016-021789

(51) Int. Cl. A61B 5/1455; A61B 5/08; A61B 5/00

(52) U.S. Cl. CPC A61B 5/1455; A61B 5/03; A61B 5/6826; A61B 5/08

(57) ABSTRACT

A biological information display apparatus includes a pulse wave acquisition part, an index derivation part, a determination part, and a display control part. The pulse wave acquisition part acquires a pulse wave signal obtained by measuring a pulse wave of a subject person along a time axis. The index derivation part derives breathing function indexes which are indexes showing states of breathing function of the subject person. The determination part determines whether at least one of the derived multiple breathing function indexes is in a predetermined range as a range of a threshold showing that the subject person gets worse. When at least one of breathing function indexes is in the predetermined range, the display control part displays progress information showing the progress of multiple breathing function indexes since becoming in the predetermined range by the at least one of breathing function indexes.

### CROSS REFERENCE TO RELATED APPLICATION

The present application is based on Japanese Patent Application No. 2016-21789 filed on Feb. 8, 2016, the disclosure of which is incorporated herein by reference.

### TECHNICAL FIELD

The present disclosure relates to a biological information display apparatus that derives and displays biological information.

### BACKGROUND ART

As described in Patent Literature 1, it is known that technology derives indexes such as oxygen saturation concentration (that is, SpO₂), rhythm of breathing, depth of breathing or the like based on a pulse wave signal sensed from a subject person, and evaluates stress applied to the subject person by following to the derived index.

### PRIOR ART LITERATURES

### Patent Literature

Patent Literature 1: JP 2006-263472 A

### SUMMARY OF INVENTION

Generally, biological information of a patient as a subject person is monitored in a medical site. A medical practitioner understands a condition of the patient based on the monitored biological information.

Therefore, the monitored biological information is preferred to report in a mode enabling how the condition of the patient has varied to be easily understood.

However, a detailed examination result by the inventors finds a difficulty of the technology described in Patent Literature 1. The difficulty that the technology only evaluates stress received by the patient and does not display the biological information in the mode enabling the progress of the condition of the patient to be easily understood is found.

That is, technology reporting the biological information is required to more easily understand the progress of the condition of the subject person. It is an object of the present disclosure to provide technology enabling a progress of the condition of the subject person to easily understand.

According to one aspect of the present disclosure, a biological information display apparatus includes a pulse wave acquisition part, an index derivation part, a determination part and a display control part.

The pulse wave acquisition part acquires a pulse wave signal obtained by measuring a pulse wave of a subject person along a time axis. The index derivation part derives a breathing function index including multiple indexes showing a state of a breathing function of the subject person based on the plus wave signal acquired by the pulse wave acquisition part.

The determination part determines whether at least one of multiple breathing function indexes derived by the derivation part is in a predetermined range. The predetermined range is predetermined as a range of a threshold showing that a condition of the subject person gets worse.

The display control part displays progress information when at least one of the multiple breathing function indexes is included in the predetermined range as the result of the determination by the determination part. The progress information shows the progress from when at least one of the multiple breathing function indexes is included in the predetermined range. Furthermore, the predetermined range is the range of the threshold showing that the condition of the subject person gets worse.

Hence, the biological information display apparatus enables the progress of the condition of the subject person to be more easily understood. Thereby, the medical practitioner more correctly understands the progress of the condition of the subject person.

### BRIEF DESCRIPTION OF DRAWINGS

The above and other aspects, features and advantages of the present disclosure will become more apparent from the following detailed description made with reference to the accompanying drawings. In the drawings:

FIG. 1 is a perspective view illustrating an appearance of a state monitoring system;

FIG. 2 is a block diagram showing a schematic configuration of a biological information display apparatus;

FIG. 3 is a flowchart showing a process procedure of a coefficient calculation process;

FIG. 4 is a flowchart showing a process procedure of an index calculation process;

FIG. 5 is a flowchart showing a process procedure of a display process according to a first embodiment;

FIG. 6 is an explanatory view explaining a mode of displaying in a normal mode in a display process of the first embodiment;

FIG. 7 is an explanatory view explaining a predetermined range;

FIG. 8A is an explanatory view showing a mode of the displaying in a first stage of an abnormal mode in the display process of the first embodiment;

FIG. 8B is an explanatory view showing a mode of the displaying in a second stage of the abnormal mode in the display process of the first embodiment;

FIG. 9 is a flowchart showing a process procedure of the display process according to a second embodiment; and

FIG. 10 is an explanatory view explaining a mode of a display in a display process of the second embodiment.

### DESCRIPTION OF EMBODIMENTS

Hereinafter, embodiments of the present disclosure will be explained in reference to the drawings.

### First Embodiment

<Biological Information Display Apparatus>

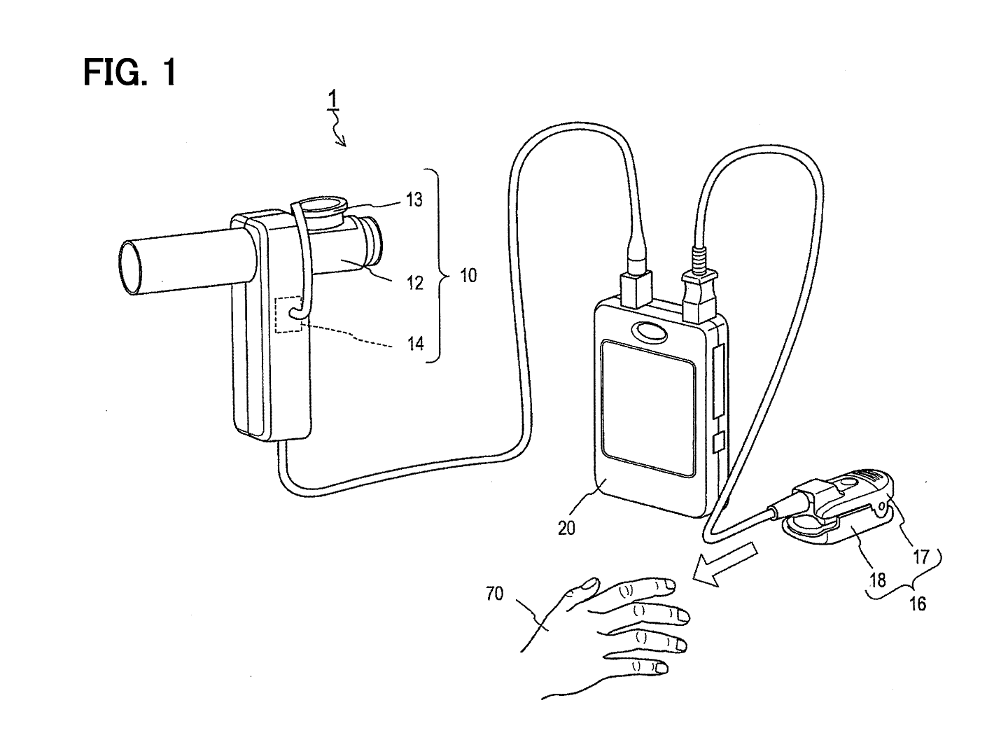

A state monitoring system 1 as shown in FIG. 1 is placed in the vicinity of a bedding of a medical institution or a patient's house. The state monitoring system 1 derives biological information of a subject person based on a pulse wave signal of the subject person and displays it. The subject person is a person attached with a pulse wave sensor 16. The subject person is a patient having a check at the medical institution or curing at home or the like, for example.

The state monitoring system 1 includes an intraoral pressure measurement apparatus 10, the pulse wave sensor 16 and a biological information display apparatus 20.

The pulse wave signal shows transition of the pulse wave. The biological information shows a vital situation of the subject person. The biological information includes a breathing function index.

The multiple breathing function indexes show a situation of the breathing function of the subject person and are different from each other. The breathing function causes blood to carry oxygen so that the oxygen is thoroughly carried to cells all over a body and is taken by a breathing organ or a circulatory organ.

The breathing function index includes an absolute value of an intrathoracic pressure, a breathing rate, oxygen saturation concentration and a pulse rate. The intrathoracic pressure is pressure inside an intrathoracic space. The breathing rate is the number of the breathing per unit time (for example, one minute). The oxygen saturation concentration is a rate of a hemoglobin binding with oxygen in an erythrocyte, in other words, is SpO₂. The pulse rate is a beating rate occurring at an artery in an entire body per unit time.

The intraoral pressure measurement apparatus 10 measures an intraoral pressure of the subject person. The intraoral pressure measurement apparatus 10 includes a cylindrical part 12 and a pressure sensor 14. The intraoral pressure is a pressure inside an oral cavity.

The cylindrical part 12 is a cylindrical member, and inhaled air and exhaled air flow in the cylindrical part 12, the inhaled air being the air inhaled by the subject person, the exhaled air being the air exhaled by the subject person. In the cylindrical part 12, a resistance setting part 13 changing a magnitude of resistance to a flowing air (that is, an exhalation) is placed.

The pressure sensor 14 of the intraoral pressure measurement apparatus 10 measures, as intraoral pressure, the pressure of the air moving inside the cylindrical part 12 by one breathing by the subject person. After the magnitude of the resistance of the cylindrical part 12 is set in multiple stages, measurement of the intraoral pressure is performed every set resistance.

The pulse wave sensor 16 is an optical pulse wave sensor including a luminous part 17 emitting light waves that have two different wavelengths different from each other and a light reception part 18 receiving the light wave from the luminous part 17. The light waves having two wavelengths emitted from the luminous part 17 are a light wave having a wavelength in an infrared region and a light wave having a wavelength of red color in a visible light region. The pulse wave sensor 16 is attached to a tip of a finger 70 of the subject person.

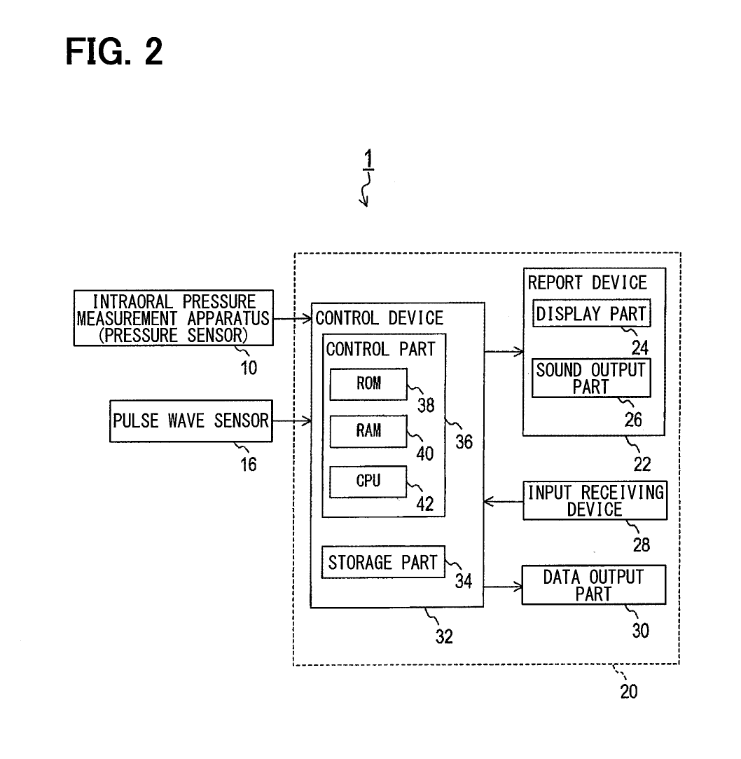

The biological information display apparatus 20 as shown in FIG. 2 includes a report device 22, an input receiving device 28, a data output part 30 and a control device 32. The biological information display apparatus 20 derives a breathing function index based on the pulse wave signal of the subject person measured by the pulse wave sensor 16 and displays it.

The report device 22 includes a display device 24 and a sound output device 26.

The display device 24 is a known device displaying an image. As the display device 24, a liquid crystal display is considered. The sound output device 26 is a known device outputting a sound. As the sound output device 26, a loudspeaker may be considered.

The input receiving device 28 is a known device receiving an input of information. The input receiving device 28 includes various input devices such as a pointing device and a switch. The pointing device includes a known touch panel.

The data output part 30 outputs to an external apparatus, a breathing function index derived by the control device 32. The external apparatus is separately placed from the biological information display apparatus 20. The external apparatus includes a portable auxiliary storage apparatus or an information process apparatus, for example. The portable auxiliary storage apparatus is an external storage apparatus that can be carried, and may be a hard disk drive or a SD memory card or the like.

The control device 32 includes a storage part 34 and a control part 36.

The storage part 34 is a rewritable nonvolatile storage device. As the storage part 34, a hard disk drive or a flash memory or the like may be considered.

The control part 36 is a known control device mainly configured from a known microcomputer including a ROM 38, a RAM 40 and a CPU 42. The ROM 38 stores data or program needed to hold a storage content even when an electric source shuts down. The RAM 40 temporally stores data. The CPU 42 executes process following to the program stored in the ROM 38 or the RAM 40.

The ROM 38 of the control part 36 stores a process program to execute various processes by the control part 36. The various processes include a coefficient calculation process, an index calculation process and a display process.

The index calculation process derives the breathing function index of the subject person based on the pulse wave signal of the subject person and stores it.

The coefficient calculation process calculates a calibration coefficient used for calculating an absolute value of the intrathoracic pressure derived by the index calculation process. The calibration coefficient is a correction coefficient converting an estimation intrathoracic pressure of the subject person to the absolute value of the intrathoracic pressure of the subject person. The estimation intrathoracic pressure shows transition of the pressure based on a relative change of amplitude of the pulse wave signal and is a relative value of the intrathoracic pressure.

The display process causes the display device 24 to display the breathing function index derived by the index calculation process.

<Coefficient Calculation Process>

The coefficient calculation process is executed after the intraoral pressure and the pulse wave signal measured from the subject person are stored in a state that the subject person breathes in an ideal breathing mode while holding the cylindrical part 12 of the intraoral pressure measurement apparatus 10 in a mouth. The ideal breathing mode is a mode of ideal breathing that measures the intraoral pressure and the pulse wave signal needed to execute the coefficient calculation, and the ideal breathing mode is a breathing pattern at rest. The breathing pattern at rest is performed by only contraction and laxity of a breathing muscle, and is not so-called effort breathing. In the other words, the ideal breathing mode is one of the modes of the breathing pattern at rest performed by the subject person and is a mode of performing breathing in a different depth multiple times.

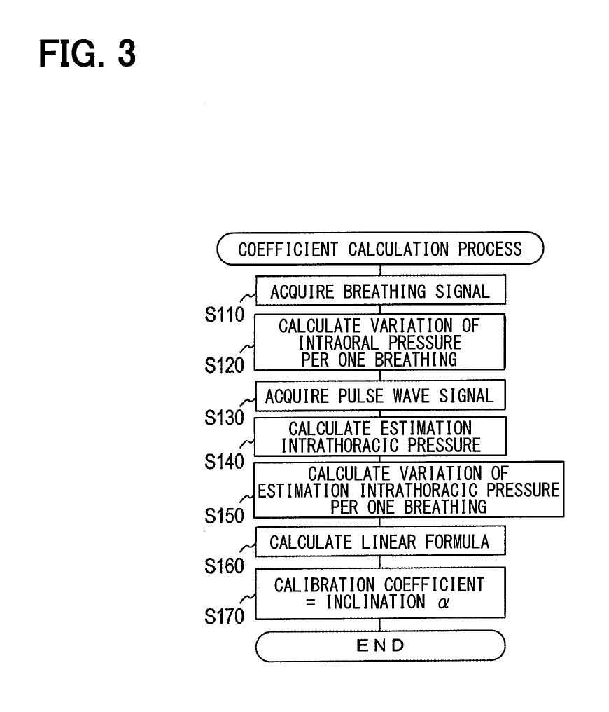

When the coefficient calculation process is executed, the control part 36 acquires the breathing signal that is measured in the ideal breathing mode and is stored in the storage part 34 as shown in FIG. 3 (S110). The breathing signal is a result measured by the pressure sensor 14 of the intraoral pressure measurement apparatus 10 while the subject person breathes in the ideal breathing mode. The breathing signal shows a transition of the intraoral pressure of the subject person in the ideal breathing mode.

The control part 36 calculates variation amount of the intraoral pressure per one breathing based on the breathing signal acquired in S110 (S120). In S120, the control part 36 calculates difference between peaks of the breathing signal each breathing and a first reference value, as the variation amount of the intraoral pressure each breathing in a transition of the intraoral pressure shown by the breathing signal. The first reference value is a preset value of the intraoral pressure. As the first reference value, value of a pressure equals to an atmosphere pressure or an intraoral pressure at an end point of the breathing may be considered.

In the coefficient calculation process, the control part 36 acquires the pulse wave signal measured in the ideal breathing mode and stored in the storage part 34 (S130). The pulse wave signal is a result measured by the pulse wave sensor 16 and shows a transition of the pulse wave while the subject person breathes in the ideal breathing mode. The pulse wave signal acquired in S130 is associated with, at least, the intraoral pressure signal acquired in S110 along a time axis.

The control part 36 calculates the estimation intrathoracic pressure based on the pulse wave signal acquired in S130 (S140). As an estimation method of the estimation intrathoracic pressure in S140, it may be considered to use a method described in JP 2002-355227 A. In an estimation of the estimation intrathoracic pressure, a first envelope connecting peaks of the amplitude of the pulse wave for one beat shown by the pulse wave signal is generated and a second envelope connecting peaks of the first envelope is generated. A difference between the first envelope and the second envelope may be calculated as the estimation intrathoracic pressure.

Furthermore, in the coefficient calculation process, the control part 36 calculates a variation amount of the estimation intrathoracic pressure per one breathing, based on the estimation intrathoracic pressure calculated in S140 (S150). Specifically, in S150 of the embodiment, the control part 36 calculates a difference between a peak of the intrathoracic pressure each breathing and a second reference value as a variation amount of the estimation intrathoracic pressure each breathing. The second reference value is a preset value of the estimation intrathoracic pressure. As the second reference value, the value of the pressure equal to the atmosphere pressure or the intrathoracic pressure at the end point of the breathing may be considered.

Furthermore, the control part 36 calculates a correspondence relation between the variation amount of the intraoral pressure and the variation amount of the estimation intrathoracic pressure in a linear formula (S160). In a calculation of the linear formula in S160, the variation amount of the intraoral pressure in S120 and the variation amount of the estimation intrathoracic pressure calculated in S150 are developed (in the other words, plotted) on a two dimensional plane, each the identical breathing. A known linear regression analysis finding a linear formula is performed to the developed variation amount of the intraoral pressure and the developed variation amount of the estimation intrathoracic pressure. As typical example of the linear regression analysis, least squares method is given.

Thereby, the linear formula showing the correspondence relation between the variation amount of the intraoral pressure and the variation amount of the estimation intrathoracic pressure is calculated.

Next, the control part 36 sets an inclination a of the linear formula calculated in S160 as a calibration coefficient (S170). That is, in S170 of the coefficient calculation process, a ratio of the variation amount of the estimation intrathoracic pressure to the variation amount of the intraoral pressure is set as the calibration coefficient. In the other words, the ratio of the variation amount of the estimation intrathoracic pressure to the variation amount of the intraoral pressure shows an inclination a of the variation amount of the intraoral pressure to the variation amount of the estimation intrathoracic pressure.

After that, the coefficient calculation process ends.

<Index Calculation Process>

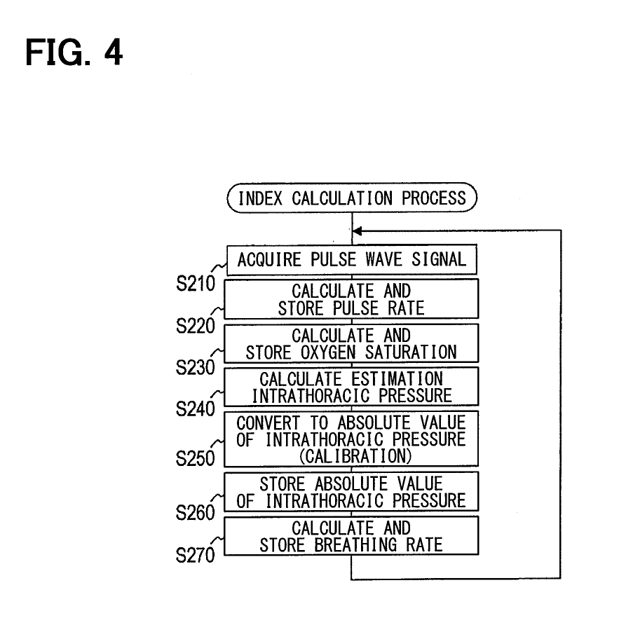

The index calculation process starts, and the control part 36 acquires the pulse wave signal measured by the pulse wave sensor 16 as shown in FIG. 4 (S210).

Next, the control part 36 calculates the pulse rate of the subject person based on the pulse wave signal acquired in S210 and associates with a present time and stores the associated pulse wave signal in the storage part 34 (S220). As the calculation method of the breathing rate according to the embodiment, a known method described in JP 2002-017694 A or JP 2001-198094 A may be used. For example, according to the method described in JP 2002-017694 A, the control part 36 performs a frequency analysis (for example, FFT) to the pulse signal and derives a frequency spectrum of the pulse wave signal. The control part 36 multiplies a peak frequency of the frequency spectrum of the derived pulse wave signal by a unit of time (that is, 60 seconds) and calculates a result of the multiplying as the pulse rate. According to the method described in JP 2001-198094 A, the control part 36 calculates a result of dividing, in which a unit of the time (that is, 60 seconds) is divided by a time interval between tops of the pulse wave signal, as the pulse rate.

In S220, the control part 36 may store the pulse rate of the subject person associated with the present time in the auxiliary storage apparatus through the data output part 30.

In the index calculation process, the control part 36 calculates the oxygen saturation concentration (that is, SpO₂) based on the pulse wave signal acquired in S210 and associates with present time and stores in the storage part 34 (S230). As the calculation method of the oxygen saturation concentration in the embodiment, a known method based on a ratio of a light reception amount of a light having a wave length in the infrared region to a light reception amount of a light having a wave length of the red color may be used, the lights being received in the light reception part 18 of the pulse wave sensor 16. The oxygen saturation concentration is calculated in the pulse wave sensor 16 and, in S230, the control part 36 may only acquire the oxygen saturation concentration calculated in the pulse wave sensor 16. In S230, the control part 36 may store the oxygen saturation concentration of the subject person associated with the present time, in the auxiliary storage apparatus through the data output part 30.

Furthermore, in the index calculation process, the control part 36 calculates the estimation intrathoracic pressure based on the pulse wave signal acquired in S210 (S240). As an estimation method of the estimation intrathoracic pressure in S240, similarly to S140 of the coefficient calculation process, a known method may be used. Therefore, a detailed explanation will be omitted here.

Next, the control part 36 calculates the absolute value of the intrathoracic pressure of the subject person (S250). Specifically, in S250, the absolute value of the intrathoracic pressure of the subject person is calculated by multiplying the estimation intrathoracic pressure calculated in S240 by the calibration coefficient set in the coefficient calculation process.

The control part 36 associates the absolute value of the intrathoracic pressure calculated in S250 with a time at the present time and stores in the storage part 34 (S260). In S260, the control part 36 may store the absolute value of the intrathoracic pressure associated with the time at the present time in the auxiliary storage apparatus through the data output part 30.

Furthermore, in the index calculation process, the control part 36 calculates the breathing rate of the subject person, associates with the present time and stores in the storage part 34 (S270). As the calculation method of the breathing rate in S270, the method described in JP 2003-339651 A may be considered, calculating an average value of a fraction of the number of the tops of the estimation intrathoracic pressure as the breathing rate.

In S270, the control part 36 may store the breathing rate of the subject person associated with the present time in the auxiliary storage apparatus through the data output part 30.

After that, the control part 36 returns the index calculation process to S210.

In the index calculation process, the control part 36 calculates, based on the pulse wave signal measured from the subject person, the absolute value of the intrathoracic pressure, the breathing rate, the oxygen saturation concentration and the pulse rate as the breathing function index, associates with the present time and stores in the storage part 34.

In the index calculation process, the absolute value of the intrathoracic pressure, the breathing rate, the oxygen saturation concentration and the pulse rate stored in the storage part 34 each may be updated by a unit of a preset set time. The set time is preferably set to be equal to or more than maximum time of a display range t described later. The set time may be 10 minutes or more, for example.

<Display Process>

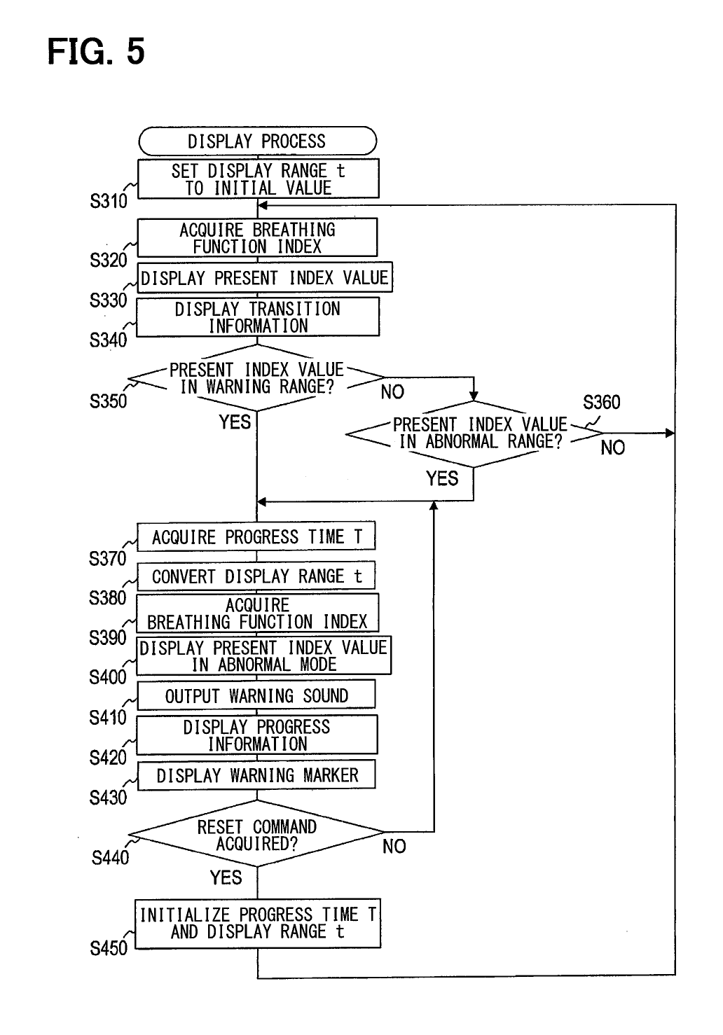

The display process starts, and the control part 36 sets the display range t to an initial value as shown in FIG. 5 (S310). The display range t is a range of a time axis in a graph showing a transition of the breathing function index. In the embodiment, the initial value may be considered to be set to a minute.

Next, the control part 36 acquires the breathing function index stored in the storage part 34 in the index calculation process (S320). Specifically, in S320, in regard to the pulse rate, the oxygen saturation concentration, the absolute value of the intrathoracic pressure and the breathing rate stored in the storage part 34, each of the pulse rate, the oxygen saturation concentration, the absolute value of the intrathoracic pressure and the breathing rate that are associated with a time within a period of a predetermined first range from the present time, is acquired. The period of the first range is a time length longer than one time length of the initial value.

The control part 36 acquires a present index value from the breathing function index acquired in S320 and displays the present index value in the display device 24 (S330). The present index value is each of the pulse rate, the oxygen saturation concentration, the absolute value of the intrathoracic pressure and the breathing rate associated with a time that is closest to the present time.

Furthermore, the control part 36 displays transition information in the display device 24, based on the breathing function index acquired in S320 (S340). The transition information is associated with each transition of multiple breathing function indexes along the time axis. Specifically, the transition information shows the transition along the time axis of the pulse rate, the oxygen saturation concentration, the absolute value of the intrathoracic pressure and the breathing rate each.

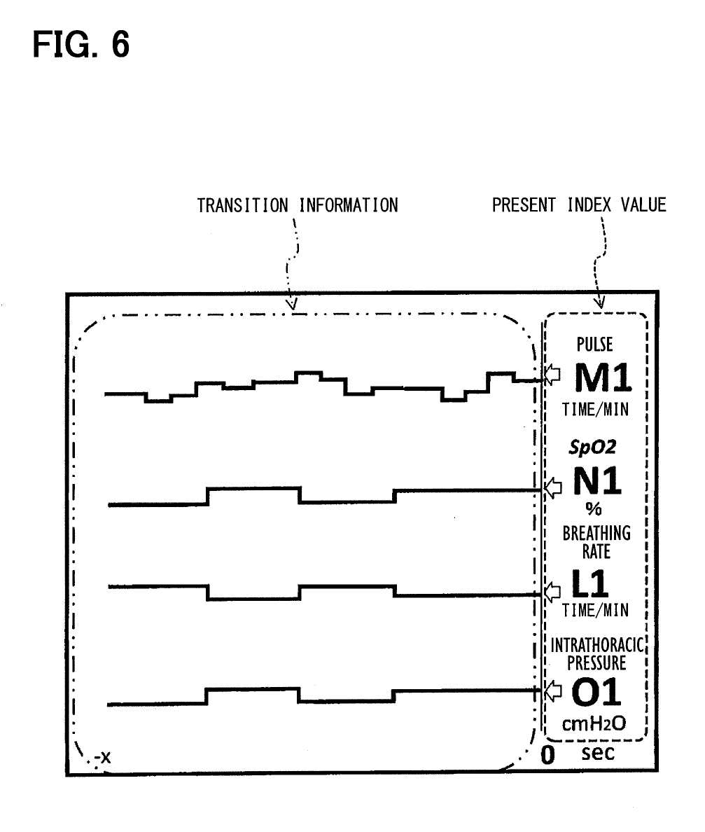

In S330, the display device 24 displays the pulse rate, the oxygen saturation concentration, the absolute value of the intrathoracic pressure and a value of the breathing rate as the present index value, as shown in FIG. 6. In S340, the display device 24 displays a graph showing transition along time axis of the pulse rate, the oxygen saturation concentration, the absolute value of the intrathoracic pressure and the breathing rate each.

In the display process, next, the control part 36 determines whether at least one of the pulse rate, the oxygen saturation concentration, the absolute value of the intrathoracic pressure or the breathing rate is in a warning range (S350). The warning range is a threshold showing that a condition of the subject person gets worse, and is predetermined.

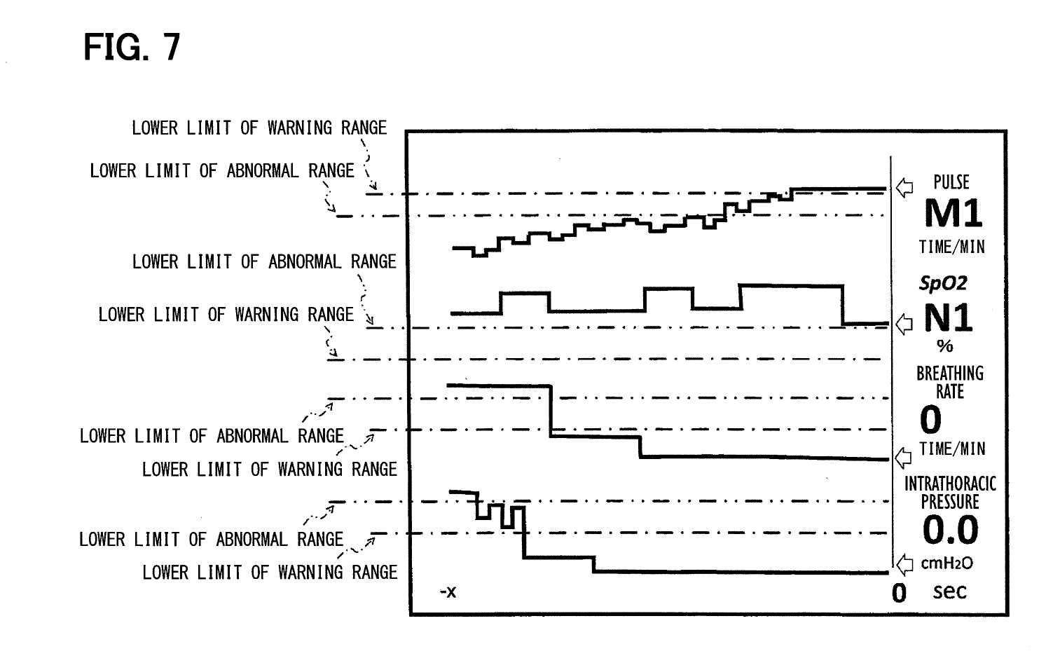

The control part 36 shifts the display process to S370 described later in detail when the at least one is in the warning range (S350: YES) as a result of the determination in S350. By contrast, the control part 36 determines whether at least one of the pulse rate, the oxygen saturation concentration, the absolute value of the intrathoracic pressure or the breathing rate as the present index value is in an abnormal range (S360) when all of the present index values are out of the warning range (S350: NO) as a result of determination in S350. The abnormal range is predetermined as a threshold showing a state that the condition of the subject person is worse than the condition of the subject person in the warning range, as shown in FIG. 7. The warning range and the abnormal range correspond to a predetermined range. The predetermined range is a threshold showing that the condition of the subject person gets worse.

The control part 36 returns the display process to S320 when all of the present index values are out of the abnormal range (S360: NO) as a result of the determination in S360. The control part 36 acquires a new present index value, displays in the display device 24, updates the transition information in the display range t of the predetermined time axis and displays in the display device 24.

By contrast, the control part 36 shifts the display process to S370 when at least one of the pulse rate, the oxygen saturation concentration, the absolute value of the intrathoracic pressure or the breathing rate as the present index value is out of the abnormal range (S360: YES) as the result of the determination in S360.

In S370, the control part 36 acquires a progress time T from when the present index value is included in the warning range or the abnormal range. Furthermore, the control part 36 changes the display range t corresponding to the progress time T (S380). Specifically, in S380, the control part 36 enlarges the display range t by a predetermined time every time that the progress time T increases by the predetermined time. The predetermined time is a predetermined time length and may be one minute, for example.

Next, the control part 36 acquires the breathing function index stored in the storage part 34 in the index calculation (S390). Specifically, in S390, in regard to each of the pulse rate, the oxygen saturation concentration, the absolute value of the intrathoracic pressure and the breathing rate stored in the storage part 34, the control part 36 acquires each of the pulse rate, the oxygen saturation concentration, the absolute value of the intrathoracic pressure and the breathing rate associated with a time within a period of a predetermined second range from the present time. The period of the second range is a time length longer than the predetermined time length of the display range t.

The control part 36 acquires the present index value from the breathing function index acquired in S390 and displays it in the display device 24 in the abnormal mode (S400). The abnormal mode is a display mode informing that the condition of the subject person gets worse. As an example of the abnormal mode, a display color of the present index value may be set to a warning color (for example, red color) or the present index value may be set to display so as to blink. In the pulse rate, the oxygen saturation concentration, the absolute value of the intrathoracic pressure and the breathing rate, only some determined to be in the warning range or the abnormal range may be displayed as the present index value displayed in the abnormal mode.

Furthermore, the control part 36 outputs a warning sound from the sound output device 26 (S410). The warning sound shows that the condition of the subject person gets worse.

Next, the control part 36 shows the transition information corresponding to a time corresponding to the set display range t, as progress information in the display device 24 (S420). The progress information shows a progress of the breathing function index from when the present index is included in the abnormal range or the warning range. The progress information in the embodiment is the transition information displayed in S420 and the present index value displayed in the abnormal mode.

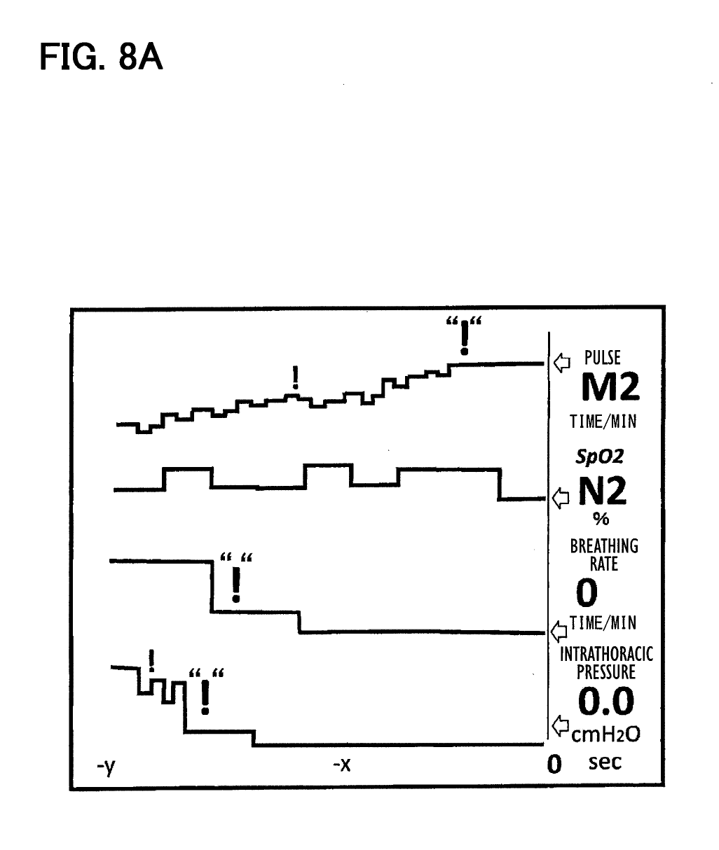

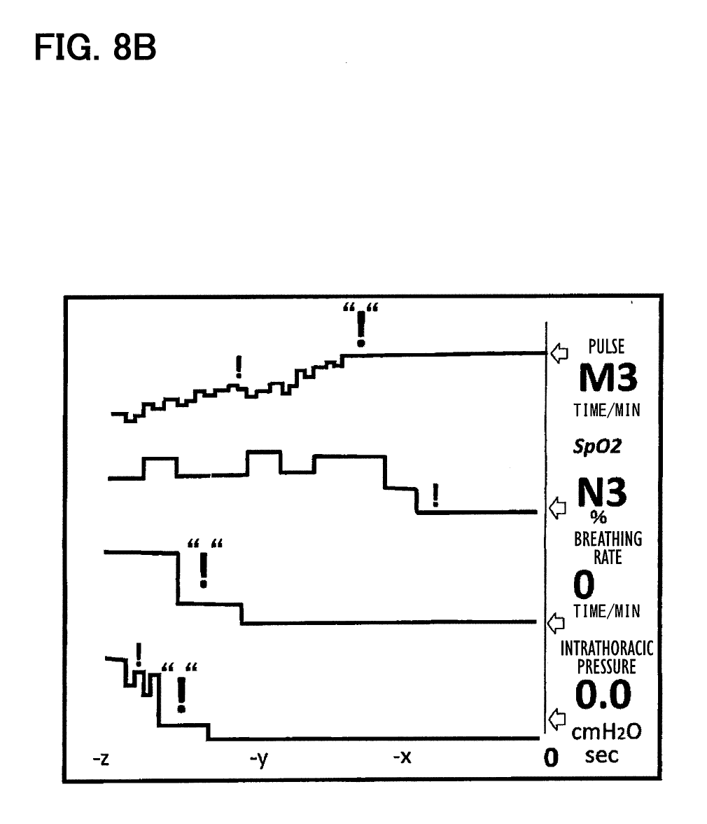

That is, in S430, the control part 36 shows in the display device 24, a graph showing a transition along the time axis of each of the pulse rate, the oxygen saturation concentration, the absolute value of the intrathoracic pressure and the breathing rate for a time in a case of enlarging the time axis of the display range t. The control part 36 shows the progress of the breathing function index from when the present index is included in the warning range or the abnormal range. Specifically, as shown in FIG. 8A, the control part 36 enlarges the time axis of the display range t from an initial value (x in FIG. 8A) to a set value (that is, y) when the set display range t is y minute. The control part 36 displays the graph of the transition information in the enlarged range in the display device 24. Furthermore, the control part 36 enlarges the time axis of the display range t from the set value (that is, y) to a set value (that is, z) as shown in FIG. 8B when the progress time T increases and the set displayed range t becomes z minute (that is, z>y). The control part 36 displays the graph of the transition information in the enlarged range in the display device 24.

Furthermore, the control part 36 shows a warning marker in the display device 24 so that the warning maker overlaps with the graph of the transition information displayed in the display device 24 (S430). The warning marker is a sign showing a moment at when the pulse rate, the oxygen saturation concentration, the absolute value of the intrathoracic pressure or the breathing rate is included in the warning range or the abnormal range. In S430, an exclamation mark may be displayed as the warning marker at the moment becoming in the warning range or the abnormal range, as shown in FIG. 8A and FIG. 8B. Another exclamation mark that is bigger than the exclamation mark displayed at the moment becoming in the warning range may be displayed as the warning mark at the moment becoming in the abnormal range.

In the display process, next, the control part 36 determines whether to acquire a reset command (S440). The reset command releases the displaying in the abnormal mode and is inputted through the input receiving device 28.

The control part 36 returns the display process to S370 when no reset command is acquired (S440: NO) as the result of the determination in S440. The control part 36 repeatedly executes S370 to S440 until acquiring the reset command. The control part 36 acquires the reset command (S440: YES), and shifts the display process to S450.

In S450, the control part 36 returns the set value of the display range t and the progress time T to the initial value.

After that, the control part 36 returns the display process to S320.

In the display process, it is determined whether at least one of the pulse rate, the oxygen saturation concentration, the absolute value of the intrathoracic pressure or the breathing rate is in the predetermined range predetermined as the range of the threshold showing that the condition of the subject person gets worse. The progress information showing the progress of the breathing function index from when becoming in the predetermined range is shown in a case of becoming in the predetermined range as the result of the determination.

### Effect of First Embodiment

(1) As described above, in the display process, a graph of the transition information and the present index value are shown. Therefore, a user such as a medical practitioner understands the state of the breathing function of the subject person.

(2) In the display process, the present index value of the breathing function index is displayed in the abnormal mode when at least one of the pulse rate, the oxygen saturation concentration, the absolute value of the intrathoracic pressure or the breathing rate as the breathing function index is included in the warning range or the abnormal range.

Hence, a user of the biological information display apparatus such as the medical practitioner may be possible to recognize that the condition of the subject person varies and also recognize which index of the breathing function indexes is included in the warning range or the abnormal range.

(3) Furthermore, in the display process, the graph of the transition information is displayed in the display device 24 with the enlarged time axis of the display range t so that the progress of the breathing function index from when the at least one of the breathing function index is included in the warning range or the abnormal range is shown.

The user such as the medical practitioner may be possible to more correctly understand the progress of the condition of the subject person and it becomes for the user to easily identify an element changing the condition, since the transition of the breathing function index from when becoming in the warning range or the abnormal range is shown.

(4) The display process shows the warning marker that shows the moment becoming in the warning range or the abnormal range.

Thereby, the user such as the medical practitioner may be possible to understand the moment becoming in the warning range or the abnormal range and it becomes for the user to easily identify the progress of the condition of the subject person.

(5) Furthermore, in the display process, the warning sound is outputted from the sound output device 26 when one of the breathing function indexes is included in the warning range or the abnormal range.

It becomes for the user such as the medical practitioner to easily understand the change of the condition of the subject person.

### Second Embodiment

The biological information display apparatus in the second embodiment is mainly different from the state monitoring system 1 in the first embodiment in regard to the display process executed by the biological information display apparatus 20. Therefore, explanations of a common configuration and a common process will be omitted by applying an identical numeral. The display process being difference point will be mainly explained.

<Display Process>

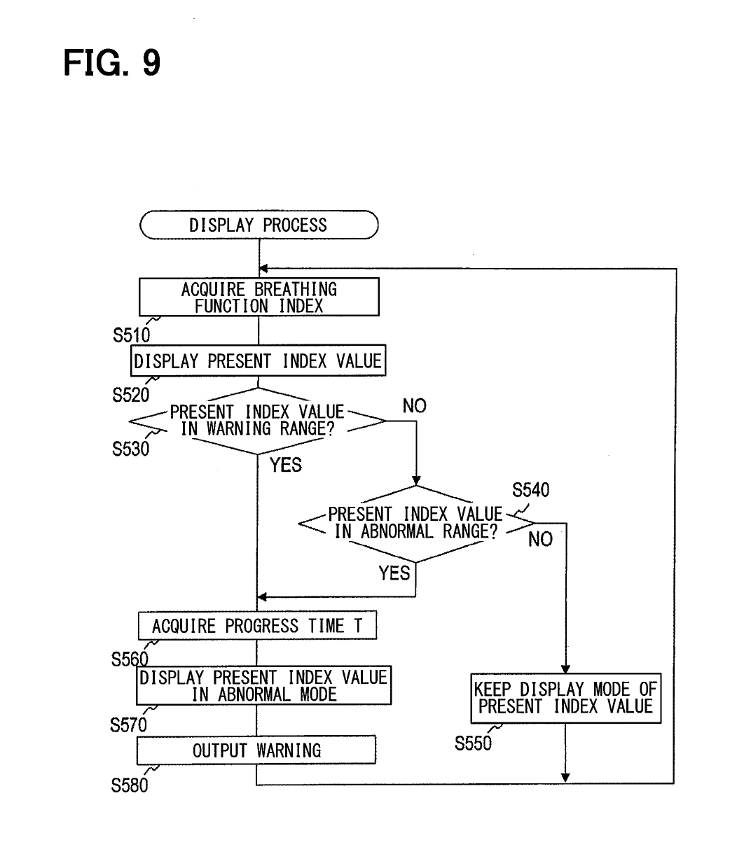

The display process of the embodiment starts, and the control part 36 acquires the breathing function index stored in the storage part 34 in the index calculation process (S510) as shown in FIG. 9. Specifically, in S510, in regard to the pulse rate, the oxygen saturation concentration, the absolute value of the intrathoracic pressure and the breathing rate that are stored in the storage part 34, each of the pulse rate, the oxygen saturation concentration, the absolute value of the intrathoracic pressure and the breathing rate as the breathing function index associated with the time in the period of the first range from the present time are acquired.

The control part 36 acquires the present index value from the breathing function index acquired in S510 and displays the present index value in the display device 24 (S520).

Furthermore, the control part 36 determines whether at least one of the pulse rate, the oxygen saturation concentration, the absolute value of the intrathoracic pressure or the breathing rate is in the warning range (S530).

The control part 36 shifts the display process to S560 described later in detail when at least one is in the warning range (S530: YES) as a result of the determination in S530.

By contrast, when all of the present index values is out of the warning range (S530: NO) as a result of the determination in S530, the control part 36 determines whether at least one of the pulse rate, the oxygen saturation concentration, the absolute value of the intrathoracic pressure or the breathing rate as the present index value is in the abnormal range (S540).

When all of the present index values are out of the abnormal range (S540: NO) as the result of the determination, the control part 36 keeps the display mode of the present index value (S550). After that, the control part 36 returns the display process to S510. The control part 36 acquires the new present index value and displays it in the display device 24.

When at least one of the pulse rate, the oxygen saturation concentration, the absolute value of the intrathoracic pressure or the breathing rate is in the abnormal range (S540: YES) as the result of the determination in S540, the control part 36 shifts the display process to S560.

In S560, the control part 36 acquires the progress time T from when at least one of multiple breathing function indexes is included in the warning range or the abnormal range.

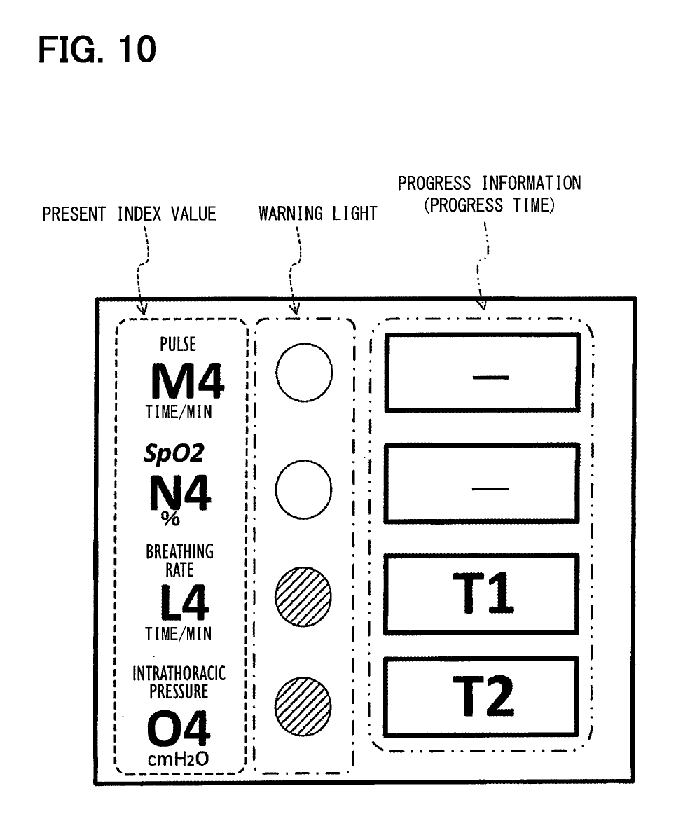

Furthermore, the control part 36 changes the display mode of the present index value displayed in the display device 24 to the abnormal mode and displays the progress time T as the progress information in the display device 24, as shown in FIG. 10 (S570). The abnormal mode is the display mode informing that the condition of the subject person gets worse. As one of the example of the abnormal mode, a display color of the present index value may be set to a warning color or the present index value may be set to display so as to blink. In the pulse rate, the oxygen saturation concentration, the absolute value of the intrathoracic pressure and the breathing rate, only some determined as in the warning range or the abnormal range may be displayed as the present index value displayed in the abnormal mode. Similarly to S550, an index determined to be out of the both of the warning range and the abnormal range may keep in the previous display mode.

Furthermore, the control part 36 outputs an warning sound from the sound output device 26 and displays warning light in the display device 24 (S580). The warning sound shows that the condition of the subject person gets worse. The warning light indicates that the condition of the subject person gets worse.

After that, the control part 36 returns the display process to S510. That is, in the display process of the embodiment, the index becoming in the warning range or the abnormal range of the present index value is displayed in the abnormal mode together with displaying the progress time T when at least one of the multiple breathing function indexes is in the warning range or in the abnormal range. Furthermore, in the display process of the embodiment, the warning light is shown and the warning sound is outputted.

### Effect of Second Embodiment

According to the display process, the progress time T from when at least one of the multiple breathing function indexes is included in the warning range or the abnormal range is displayed as the progress information.

Therefore, the biological information display apparatus 20 may enable the user such as the medical practitioner to easily recognize the progress from when the condition of the subject person becomes worse.

Particularly, the display process of the embodiment may be possible to narrow the display area of the display device 24 since the information displayed in the display device 24 is simplified.

### Other Embodiments

Embodiments of the present disclosure are explained. However, the present disclosure is not limited to the embodiments described above, and can be modified as appropriate. It may be possible to perform the present disclosure by a verified combination in the field not over the point of present disclosure.

(1) In the embodiment, the breathing function index may be at least two of the absolute value of the intrathoracic pressure, the breathing rate, the oxygen saturation concentration or the pulse rate though the absolute value of the intrathoracic pressure, the breathing rate, the oxygen saturation concentration and the pulse rate are supposed as the breathing function index. The breathing function index may include at least one of a breathing rhythm and a presence of the breathing.

The breathing rhythm is an index showing a periodicity with a depth and the moment of the breathing. The presence of the breathing is an index showing whether to breathe.

(2) In the embodiment, a target outputted to and stored in the auxiliary storage apparatus through the data output part 30 may include the progress information, though each breathing function index is supposed as the target outputted to and stored in the auxiliary apparatus through the data output part 30.

(3) A part of or all functions executed by the control device 32 in the embodiment is configured from one or more IC or the like as hardware.

(4) In the embodiment, though the ROM 38 stores the program, the storage medium storing the program is not limited to the configuration and a non-transitory tangible storage medium such as a semiconductor memory may store the program.

(5) The control device 32 may execute the program stored in the non-transitory tangible storage medium. A method corresponding to the program is achieved by executing the program.

(6) The embodiment of the present disclosure includes an aspect that a part of the configuration of the embodiment is omitted. The embodiment of the present disclosure includes an aspect configured from an appropriate combination of the embodiment and the modification. The embodiment of the present disclosure includes any aspect considered in the field not over the point of present disclosure identified by the wording described in the scope of the present disclosure.

(7) The reference used to explain the embodiment is used to easily understand the present disclosure and the use of the reference does not intend to limit to the technical scope of the present disclosure though the reference is appropriately used in the scope of the present disclosure.

[Correspondence Relation]

The function achieved by executing S210 of the index calculation process corresponds to a pulse wave acquisition part. The function achieved by executing S220 to S270 corresponds to an index derivation part. The function achieved by executing S320, S350, S360, S510, S530, and S540 of the display process corresponds to a determination part. The function achieved by executing S330, S340, S370 to S400, S420, S520, S550 to S580 corresponds to a display control part.

The function achieved by executing S410, S450, and S570 of the display process corresponds to a report control part. The function achieved by executing S140 of the coefficient calculation process corresponds to an intrathoracic pressure calculation pressure part. The function achieved by executing S110 corresponds to a breathing signal acquisition part. The function achieved by executing S120, S150 to S170 corresponds to a coefficient calculation part.

The function achieved by executing S220, S230, S260, and S270 of the index calculation process corresponds to an output part.

What is claimed is:

1. A biological information display apparatus comprising:
a pulse wave acquisition part that acquires a pulse wave signal obtained by measuring a pulse wave of a subject person along a time axis;
an index derivation part that derives a breathing function index including a plurality of indexes showing a state of a breathing function of the subject person, based on the pulse wave signal acquired by the pulse wave acquisition part;
a determination part that determines whether at least one of the plurality of the breathing function indexes derived by the index derivation part is in a determined range predetermined as a range of a threshold showing that a condition of the subject person gets worse; and
a display control part that displays progress information showing progress of the plurality of the breathing function indexes from when the at least one of the plurality of the breathing function indexes is included in the predetermined range in a case that the at least one of the plurality of the breathing function indexes is included in the predetermined range as a result of determination by the determination part.

2. The biological information display apparatus according to claim 1, wherein:
the display control part displays transition information associated along the time axis with each transition of the plurality of the breathing function indexes, as the progress information.

3. The biological information display apparatus according to claim 2, wherein:
the display control part displays the transition information in the display range along the predetermined time axis when the all of the plurality of the breathing function indexes are outside the predetermined range as the result of the determination by the determination part; and
the display control part enlarges the time axis of the display range and displays the transition information when the at least one of the plurality of the breathing function indexes is included in the predetermined range as the result of the determination so that the progress of the plurality of the breathing function indexes from when the at least one of the plurality of the breathing function indexes is included in the predetermined range is shown.

4. The biological information display apparatus according to claim 1, wherein:
the display control part displays the progress time from when the at least one of the plurality of the breathing function indexes is included in the predetermined range as the progress information.

5. The biological information display apparatus according to claim 1, further comprising:
a report control part that reports that the condition of the subject person gets worse when the at least one of the plurality of the breathing function indexes is included in the predetermined range as the result of the determination by the determination part.

6. The biological information display apparatus according to claim 1, further comprising:
an intrathoracic pressure calculation part that calculates an intrathoracic pressure of the subject person based on the pulse wave acquired by the pulse wave acquisition part;
a breathing acquisition part that acquires a breathing signal, which shows a magnitude of an intraoral pressure of the subject person at when the subject person breathes in a different depth along the time axis, the breathing signal being associated with the pulse wave signal acquired by the pulse wave acquisition part along the time axis; and
a coefficient calculation part that calculates as a calibration coefficient, a ratio of the variation amount from a preset second reference value of an amplitude of the pulse wave signal to a variation amount from a preset first reference value of the intraoral pressure shown by the breathing signal, based on the breathing signal acquired by the breathing acquisition part and on the pulse wave acquisition signal acquired by the pulse wave acquisition part,
wherein:
the index derivation part derives an absolute value of the intrathoracic pressure of the subject person as one of the plurality of the breathing function indexes, the absolute value of the intrathoracic pressure of the subject person being a result of multiplying an estimation intrathoracic pressure by the calibration coefficient, the estimation intrathoracic pressure being a relative value of the intrathoracic pressure estimated based on the pulse wave signal acquired by the pulse wave acquisition part, the calibration coefficient being calculated by the coefficient calculation part.

7. The biological information display apparatus according to claim 1, wherein:
the index derivation part derives at least one of a breathing rate, a breathing rhythm, or a presence of a breathing of the subject person, as one of the plurality of the breathing function indexes.

8. The biological information display apparatus according to claim 1, wherein:
the pulse wave acquisition part acquires the pulse wave signal from an optical pulse wave sensor measuring the pulse wave signal of the subject person based on a result of receiving light after emitting light waves that have two wavelengths different from each other; and
the index derivation part derives an oxygen saturation concentration of the subject person as one of the plurality of the breathing function indexes, based on the pulse wave signal.

9. The biological information display apparatus according to claim 1, wherein:
the index derivation part derives the pulse rate of the subject person as one of the plurality of the breathing function indexes.

10. The biological information display apparatus according to claim 1, wherein:
the pulse wave acquisition part acquires the pulse wave signal from the pulse wave sensor attached to a tip of a finger of the subject person.

11. The biological information display apparatus according to claim 1, further comprising:
an output part that outputs to an external apparatus, the at least one of the plurality of the breathing function indexes or the progress information.

12. The biological information display apparatus according to claim 11, wherein:
the output part outputs the at least one of the plurality of the breathing function indexes or the progress information to a portable auxiliary storage apparatus as the external apparatus.
